(19) United States
(12) Patent Application Publication
(10) Pub. No.: US 2014/0155928 A1
(43) Pub. Date: Jun. 5, 2014

(54) LONG-TERM OBSTRUCTION DEVICE

(71) Applicants: Izhak Fabian, Kfar Truman (IL); Nir Altman, Kibbutz Kfar Etzion (IL); Steven Haas, Kochav Yair (IL); Yoav Hirsch, Modiin (IL)

(72) Inventors: Izhak Fabian, Kfar Truman (IL); Nir Altman, Kibbutz Kfar Etzion (IL); Steven Haas, Kochav Yair (IL); Yoav Hirsch, Modiin (IL)

(21) Appl. No.: 13/705,359

(22) Filed: Dec. 5, 2012

(51) Int. Cl. A61B 17/12

(52) U.S. Cl. CPC A61B 17/12022
USPC 606/192

(57) ABSTRACT

An obstruction device including a proximal obstruction balloon and a distal obstruction balloon mounted on a shaft, wherein a neck portion of said shaft provides a gap between a distal end of said proximal obstruction balloon and a proximal end of said distal obstruction balloon.

### FIELD OF THE INVENTION

The present invention generally relates to devices for obstructing or reducing flow through a body lumen, in particular for obstructing or reducing flow of gastric contents across the pyloric valve.

### BACKGROUND OF THE INVENTION

In the prior art, when an occlusion of the pylorus is required in the course of a gastroplasty procedure or in a procedure that involves the duodenum, the surgeon staples the pylorus shut (in the stomach) and this is a short term occlusion to allow the duodenum to recover from an operation. Transpyloric devices have also been proposed, which may partially and/or intermittently obstruct the pylorus, thereby decreasing the flow of gastric contents into the duodenum.

However, there are some chronic patients who require long-term obstruction of the pylorus. Long-term obstruction is problematic. The gastrointestinal (GI) environment applies forces that tend to move the plug out of place over time, including normal peristaltic movement and other movements, such as coughing or vomiting. The plug must withstand the chemical environment, too. Accordingly, a chronic pyloric plug must be fixed stronger around the pylorus and have a tighter fit than temporary solutions.

### SUMMARY OF THE INVENTION

The present invention seeks to provide an improved device for obstructing or reducing flow through a body lumen, in particular for obstructing or reducing flow of gastric contents across the pyloric valve (pylorus), as is described more in detail hereinbelow. The device is particularly useful in a transoral gastrointestinal procedure, but the invention is not limited to transoral gastroplasty, and may be used in other laparoscopic, endoscopic, or natural orifice procedures in other body lumens.

The obstruction device (also called pyloric plug or just “plug”) is designed to block the flow of enteric contents (food, fluids, etc.) from the stomach to the duodenum. The plug is designed to be fully operative over a long time, such as but not limited to, between six months and many years. The device can be removed, if desired, and can also be re-implanted.

The present invention is particularly useful to stop the flow of stomach contents in to the proximal gut which includes the duodenum and the initial part of the jejunum. Such a need arises, for example, after creating an alternative path of flow through a gastro-jejunum anastomosis which bypasses the proximal gut. There could be other cases when this need arises, such as after surgery in the duodenum area or in the pancreas or bile outputs to the duodenum. Another indication could be the need to operate endoscopically on the stomach with an inflated stomach. In this case, the plug keeps the inflating air in the stomach and it does not bloat the intestine.

In a more specific example, the plug can be used in a method for creating an anastomosis between a stomach and a portion of a small intestine, wherein the long-term plug is used to control passage of stomach contents through the pylorus during and after creation of the anastomosis. For example, before the anastomosis has been created, the plug would allow passage of material therethrough, but after creation of the anastomosis the pylorus plug would either completely block flow (so that material only flows through the anastomosis) or partially block flow (so that material can flow through both the plug and the anastomosis). Such a method is described in U.S. patent application Ser. No. 13/484498, filed 31 May 2012 (the plug of the present invention was not described therein).

The plug is inserted by a delivery system and may be retrieved easily, if desired.

There is thus provided in accordance with an embodiment of the present invention an obstruction device including a proximal obstruction balloon and a distal obstruction balloon mounted on a shaft, wherein a neck portion of said shaft provides a gap between a distal end of said proximal obstruction balloon and a proximal end of said distal obstruction balloon.

In accordance with an embodiment of the present invention the distal obstruction balloon includes one or more anchoring members.

### BRIEF DESCRIPTION OF THE DRAWINGS

The present invention will be understood and appreciated more fully from the following detailed description taken in conjunction with the drawings in which:

FIGS. 1A and 1B are simplified pictorial illustrations of an obstruction device, respectively after and before inflation thereof, constructed and operative in accordance with an embodiment of the present invention.

### DETAILED DESCRIPTION OF EMBODIMENTS

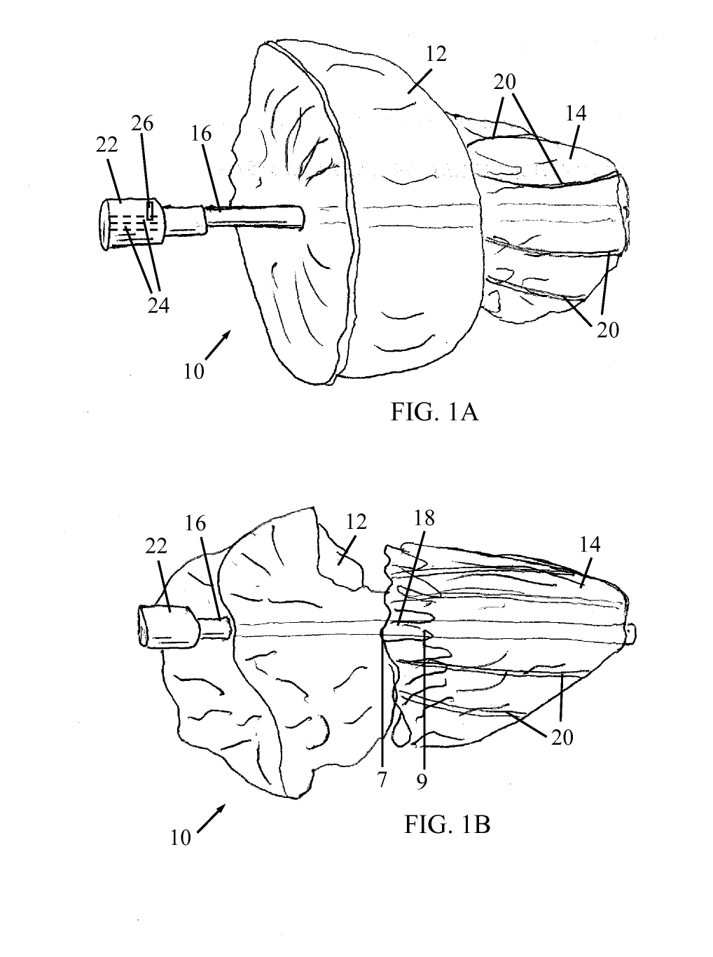

Reference is now made to FIGS. 1A-1B, which illustrate an obstruction device 10, constructed and operative in accordance with an embodiment of the present invention.

Obstruction device 10 includes a proximal obstruction balloon 12 and a distal obstruction balloon 14 mounted on a shaft 16. In the deflated configuration in FIG. 1B, one can see that a relatively short portion of shaft 16, referred to as neck 18 or neck portion 18, provides a gap between the distal end 7 of proximal obstruction balloon 12 and the proximal end 9 of distal obstruction balloon 14. Neck 18 can have different lengths and thicknesses depending on the application; for example, the dimensions of neck 18 are correlated to the usual width of the pylorus muscle. Neck 18 is preferably dimensioned so that no long-term pressure is applied to the pylorus.

The proximal obstruction balloon 12 is arranged to fit in the stomach, whereas the distal obstruction balloon 14 is arranged to fit in the duodenum. When inflated, balloons 12 and 14 expand towards the pylorus and put pressure from opposite sides on the pylorus, thus fixing the plug 10 in place. The balloons 12 and 14 may be inflated with saline, air or other fluid, using a catheter (not shown) that is passed through the working channel of an endoscope (not shown). Each balloon design expands in a required direction so that as it expands, it increases pressure on the pylorus.

In accordance with an embodiment of the present invention, distal obstruction balloon 14 includes a plurality of internal or external anchoring arms 20. In a preferred embodiment, anchoring arms 20 are constructed from folds in balloon 14. One purpose of arms 20 is to help anchor the device against the pylorus in the duodenum. Another purpose is to create a non-uniform surface for pushing against tissue (e.g., the distal side of the pylorus). The non-uniform surface may help prevent creating constant pressure against the duodenal side of the pylorus; constant pressure has the disadvantageous risk of causing a sore, like a pressure sore, on the tissue.

The anchoring arms 20 may be useful for maintaining the balloon 14 in place in the duodenum, because they can maintain anchoring forces on the duodenum walls even in the presence of variable pressure on the duodenum walls. Since there is generally less abrasion on the distal side of the pylorus, a perfect seal is not necessary, and anchoring is more important than sealing. On the stomach side, balloon 12 may have a uniform inflated shape that blocks flow from the stomach. Alternatively, balloon 12 may also be provided with anchoring arms.

In an alternative embodiment, anchoring arms 20 may be arcuate loops of a flexible yet strong material suitable for anchoring against the intestinal walls, such as but not limited to, NITINOL or stainless steel alloy.

In accordance with an embodiment of the present invention, a proximal connector 22 is mounted on shaft 16. Connector 22 may be held by a grasping tool when inserting the device into the duodenum or other lumen and for retrieval therefrom. Connector 22 is a fluid connector used to inflate the balloons 12 and 14. Connector 22 may be connected to a delivery catheter (not shown), through which fluid (saline, air or other fluid) is introduced through connector 22 to inflate the balloons, and conversely to withdraw fluid from the balloons to deflate them. Connector 22 may have just one lumen 24, or as shown in FIG. 1A, may be a multi-lumen connector with lumens 24 for fluid communication with the balloons, wherein lumens 24 pass through hollow shaft 16. Each lumen 24 is dedicated to inflating/deflating its own balloon. Each balloon 12 and 14 can be inflated separately, and with different pressure. This may be useful for different attachment strengths and pressures on the pylorus tissue.

In one embodiment, balloons 12 and 14 are interconnected such that if the proximal obstruction balloon 12 deflates, so does the distal obstruction balloon 14, whereas if the distal obstruction balloon 14 deflates, the proximal obstruction balloon 12 does not deflate (unless deflated separately).

In one embodiment, shaft 16 is hollow and open at its distal end. Connector 22 may include a valve 26 (FIG. 1A) for selectively opening or closing the passage to the pylorus. Valve 26 may be remotely controlled by an external device (such as being electronically or magnetically controlled), or may be controlled manually by a suitable push-pull wire and the like.

It will be appreciated by persons skilled in the art that the present invention is not limited by what has been particularly shown and described hereinabove. Rather the scope of the present invention includes both combinations and subcombinations of the features described hereinabove as well as modifications and variations thereof which would occur to a person of skill in the art upon reading the foregoing description and which are not in the prior art.

What is claimed is:

1. An obstruction device comprising:
a proximal obstruction balloon and a distal obstruction balloon mounted on a shaft, wherein a neck portion of said shaft provides a gap between a distal end of said proximal obstruction balloon and a proximal end of said distal obstruction balloon.

2. The obstruction device according to claim 1, wherein said distal obstruction balloon comprises a plurality of anchoring arms.

3. The obstruction device according to claim 2, wherein said anchoring arms are constructed from folds in said distal obstruction balloon.

4. The obstruction device according to claim 2, wherein said anchoring arms are operative to create a non-uniform surface for pushing against tissue.

5. The obstruction device according to claim 1, comprising a proximal connector mounted on said shaft.

6. The obstruction device according to claim 5, wherein said connector comprises a plurality of lumens in fluid communication with said proximal and distal obstruction balloons, wherein said lumens pass through said shaft.

7. The obstruction device according to claim 6, wherein each of said lumens is in dedicated fluid communication with a particular one of said proximal and distal obstruction balloons, so that each of said balloons is separately inflatable with different pressure.

8. The obstruction device according to claim 1, wherein said shaft is hollow and open at its distal end, and a valve is in fluid communication with said shaft.

9. The obstruction device according to claim 1, wherein said proximal obstruction balloon and said distal obstruction balloon are interconnected such that if said proximal obstruction balloon deflates, said distal obstruction balloon also deflates, whereas if said distal obstruction balloon deflates, said proximal obstruction balloon does not deflate.
